(19) United States
(12) Patent Application Publication
(10) Pub. No.: US 2007/0143138 A1
(43) Pub. Date: Jun. 21, 2007

(54) Prescription care support services

(76) Inventors: S. Michael Ross, Merion Station, PA (US); Steve Zirkel, Dunwoody, GA (US)

Correspondence Address: PERKINS COIE LLP;PATENT-SEA, P.O. BOX 1247, SEATTLE, WA, 98111-1247, US

(21) Appl. No.: 11/311,917

(22) Filed: Dec. 19, 2005

(51) Int. Cl. G06Q 10/00; A61B 5/00

(52) U.S. Cl. 705/2; 600/300

(57) ABSTRACT

The present invention relates to prescription management systems and associated methods, including prescription management methods in computing environments that provide prescription fulfillment management, related care support services, patient opinion information collection, and the ability to interface a prescription management system with other related systems. Certain embodiments of the invention are directed toward a method of collecting prescription related data for multiple patients, where each patient can be associated with one or more prescriptions. The method can further include contacting each patient and providing information to at least one of the patients about fulfillment of the one or more prescriptions, about care support services related to the one or more prescriptions, and/or to request patient opinion information. In other embodiments, a prescription management method can include receiving raw prescription related data and providing formatted prescription related data for use in a prescription management element.

### TECHNICAL FIELD

The present invention relates to prescription management systems and associated methods, including prescription management methods in computing environments that provide prescription fulfillment management, related care support services, patient opinion information collection, and the ability to interface a prescription management system with other related systems.

### BACKGROUND

Managing the fulfillment of prescription medications and other prescription related services can be time-consuming and expensive for pharmaceutical providers. For example, pharmacist not only fill prescriptions, but must also be concerned with drug interaction issues, patient counseling, generic substitutions, dosage adherence issues, and drug recalls. Additionally, many pharmacists provide care support services related to various prescription medications, such as high-risk side effect surveillance and related healthcare education. Furthermore, in many instances the pharmacists acts as an interface between the patient, physician, and other entities regarding various prescription matters, including prescription renewals, insurance issues, and various patient concerns.

For many patients, customer service is an important factor in choosing a pharmacy and can also affect the overall quality of healthcare received by the patient. Accordingly, it is important for the pharmacist to understand which services are important to the pharmacist's patients and how the patients perceive the pharmacy is performing in various areas. Additionally, it is important that the pharmacist and the pharmacist's staff have the resources and time necessary to provide quality service. Therefore, it can be important to insure that the pharmacist and the pharmacist's staff are used effectively and efficiently in order to provide quality service and quality healthcare to the pharmacist's patients.

### BRIEF DESCRIPTION OF THE DRAWINGS

FIG. 1 is a block diagram that illustrates a computing environment suitable for implementing a prescription management process in accordance with embodiments of the invention.

FIG. 2 is a flow diagram that illustrates a prescription management process related to prescription fulfillment in accordance with various embodiments of the invention.

FIG. 3 is a flow diagram that illustrates a portion of a prescription management process related to prescription fulfillment in accordance with selected embodiments of the invention.

FIG. 4 is a flow diagram that illustrates a portion of a prescription management process related to prescription fulfillment in accordance with other embodiments of the invention.

FIG. 5 is a flow diagram that illustrates a portion of a prescription management process related to prescription fulfillment in accordance with still other embodiments of the invention.

FIG. 6 is a flow diagram that illustrates a portion of a prescription management process related to prescription fulfillment in accordance with yet other embodiments of the invention.

FIG. 7 is a flow diagram that illustrates a portion of a prescription management process related to prescription fulfillment in accordance with still other embodiments of the invention.

FIG. 8 is a flow diagram that illustrates a portion of a prescription management process related to prescription fulfillment in accordance with still other embodiments of the invention.

FIG. 9 is a flow diagram that illustrates a prescription management process related to providing information requesting patient opinion data associated with a medication in accordance with embodiments of the invention.

FIG. 10 is a flow diagram that illustrates a portion of a prescription management process that is related to providing information that reminds a patient to refill a prescription and requests patient opinion information associated with a medication in accordance with various embodiments of the invention.

FIG. 11 is a flow diagram that illustrates a portion of a prescription management process that is related to providing information that reminds a patient to refill a prescription and requests patient opinion information associated with a medication in accordance with other embodiments of the invention.

FIG. 12 is a flow diagram that illustrates a prescription management process for providing information regarding care support services related to prescription medication in accordance with embodiments of the invention.

FIG. 13 is a flow diagram that illustrates a portion of a prescription management process for providing information regarding care support services related to prescription medication in accordance with various embodiments of the invention.

FIG. 14 is a flow diagram that illustrates a portion of a prescription management process for providing information regarding care support services related to prescription medication in accordance with other embodiments of the invention.

FIG. 15 is a flow diagram that illustrates a portion of a prescription management process for providing information regarding care support services related to prescription medication in accordance with still other embodiments of the invention.

FIG. 16 is a flow diagram that illustrates a process for providing formatted prescriptions related data for use in a prescription management element in accordance with certain embodiments of the invention.

FIG. 17 is a partially schematic illustration of a prescription management system in accordance with embodiments of the invention.

### DETAILED DESCRIPTION

In the following description, numerous specific details are provided in order to give a thorough understanding of embodiments of the invention. One skilled in the relevant art will recognize, however, that the invention may be practiced without one or more of the specific details, or with other methods, components, materials, etc. In other instances, well known structures, materials, or operations are not shown or described in order to avoid obscuring aspects of the invention.

References throughout the specification to “one embodiment” or “an embodiment” means that a particular feature, structure, or characteristic described in connection with the embodiment is included in at least one embodiment of the present invention. Thus, the appearances of the phrase “in one embodiment” or “in an embodiment” in various places throughout the specification are not necessarily all referring to the same embodiment. Furthermore, the particular features, structures, or characteristics may be combined in any suitable manner in one or more embodiments.

The present invention is directed generally toward prescription management systems and associated methods, including prescription management methods in computing environments that provide prescription fulfillment management, related care support services, patient opinion information collection, and the ability to interface a prescription management system with other related systems. Aspects of the invention are directed toward a method for managing prescription medication fulfillment that includes collecting prescription related data for each one of multiple patients. Each patient can be associated with one or more prescriptions. The prescription related data can include a patient identification and/or one or more drug names associated with the one or more prescriptions. The method can further include contacting at least one of the patients and providing information about fulfillment of the patient's one or more prescriptions. In certain embodiments, the method can further include providing one or more response options regarding the fulfillment of the patient's one or more prescriptions and receiving an input indicating a selection of at least one of the one or more response options. In selected embodiments, the method for managing prescription medication fulfillment can include a method in a computing environment. In other embodiments, the method can include providing information to at least one of the patients via phone.

Other aspects of the invention are directed toward a method for providing care support services related to prescription medication, wherein the method includes collecting prescription related data for each one of multiple patients. Each patient can be associated with one or more prescriptions. The prescription related data can include a patient identification and/or one or more drug names associated with the one or more prescriptions. The method can further include contacting at least one of the patients and providing a care support service to the patient related to the patient's one or more associated prescriptions. In certain embodiments, the method can include providing one or more response options related to the care support service and receiving an input indicating a selection of at least one of the one or more response options. In selected embodiments, the method for providing care support services related to prescription medication can include a method in a computing environment. In other embodiments, the method can include providing a care support service to the patient via phone.

Still other aspects of the invention are directed toward a method of collecting patient opinion information associated with a prescription medication, wherein the method includes collecting prescription related data for each one of multiple patients. Each patient can be associated with one or more prescriptions. The prescription related data can include a patient identification and/or one or more drug names associated with the one or more prescriptions. The method can further include contacting at least one of the patients and providing one or more response options to the patient regarding a request for patient opinion information. The method can still further include receiving an input from the patient indicating a selection of at least one of the one or more response options and providing an output based on the patients' selection of at least one of the one or more response options. In certain embodiments, the method of collecting patient opinion information includes a method in a computing environment.

Yet other aspects of the invention are directed toward a method for providing formatted prescription related data for use in a prescription management element that includes collecting raw prescription related data for each one of multiple patients. Each patient can be associated with one or more prescriptions. The prescription related data can include a patient identification and/or one or more drug names associated with the one or more prescriptions. The method can further include processing the raw prescription related data for each one of the multiple patients to produce formatted prescription related data. The method can still further include providing the formatted prescription related data for each one of the multiple patients to a prescription management element. In selected embodiments, the method can further include collecting drug related information. In certain embodiments, the method for providing formatted prescription related information includes a method in a computing environment.

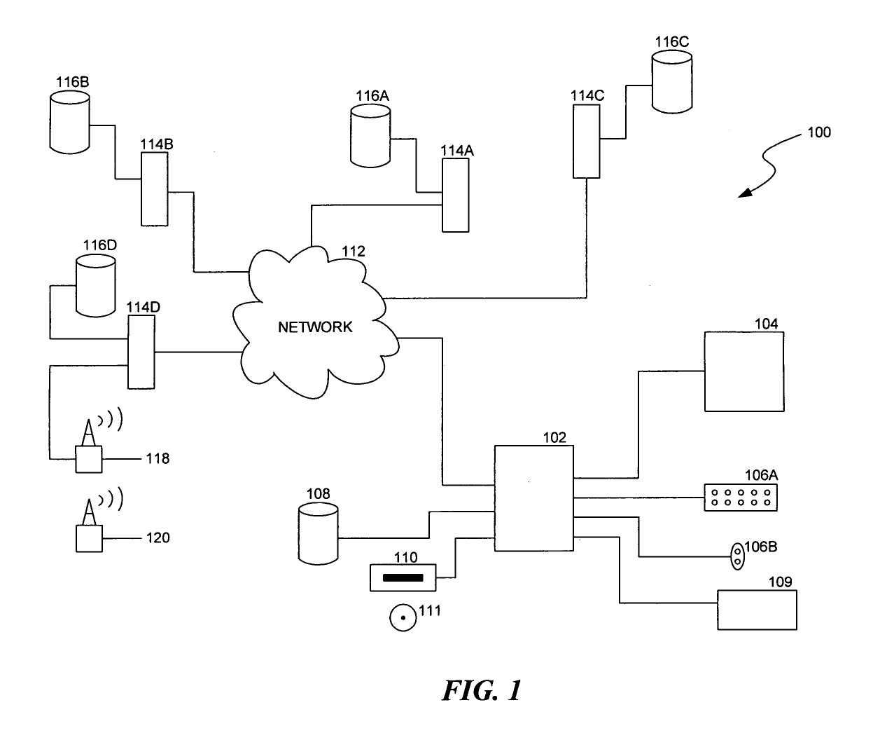

FIG. 1 is a block diagram that illustrates a computing environment suitable for implementing a process related to a prescription management system in accordance with embodiments of the invention. The computing environment 100 can include a computing or computer system 102 that can be operably connected or coupled to a display 104 and one or more input devices, for example, a keyboard 106a and a pointing device 106b (e.g., a mouse). Additionally, the computer system 102 can communicate with one or more storage devices (e.g., a hard drive 108 with one or more databases) and one or more devices 110 for reading other types of computer readable mediums (e.g., devices for reading disks 111). The computing system 102 can also communicate directly with other devices 109, for example, a phone and/or fax system, or with other devices or systems via a network 112 (e.g., via the Internet). For example, in the illustrated embodiment the computer system 102 can communicate with other computer systems 114a-d and/or other databases 116a-d via the network 112. Additionally, in selected embodiments the computing system can communicate with wireless computing devices 120 (e.g., a personal data assistant) via a wireless transmitter/receiver (e.g., a service provider connected to the internet). In other embodiments, the computing environment can have other arrangements, including more, fewer, and/or different components.

For example, the computing device or environment on which the system is implemented may include a central processing unit, memory, input devices (e.g., keyboard and pointing devices), output devices (e.g., display devices), and storage devices (e.g., disk drives). The memory and storage devices are computer-readable media that may contain instructions that implement the system. In addition, the data structures and message structures may be stored or transmitted via a data transmission medium, such as a signal on a communication link. Various communication links may be used, such as the Internet, a local area network, a wide area network, a point-to-point dial-up connection, a cell phone network, and so on.

Embodiments of the system may be implemented in various operating environments that include personal computers, server computers, hand-held or laptop devices, multiprocessor systems, microprocessor-based systems, programmable consumer electronics, digital cameras, network PCs, minicomputers, mainframe computers, distributed computing environments that include any of the above systems or devices, and so on. The computer systems may be cell phones, personal digital assistants, smart phones, personal computers, programmable consumer electronics, digital cameras, and so on.

The system may be described in the general context of computer-executable instructions, such as program modules, executed by one or more computers or other devices. Generally, program modules include routines, programs, objects, components, data structures, and so on that perform particular tasks or implement particular abstract data types. Typically, the functionality of the program modules may be combined or distributed as desired in various embodiments.

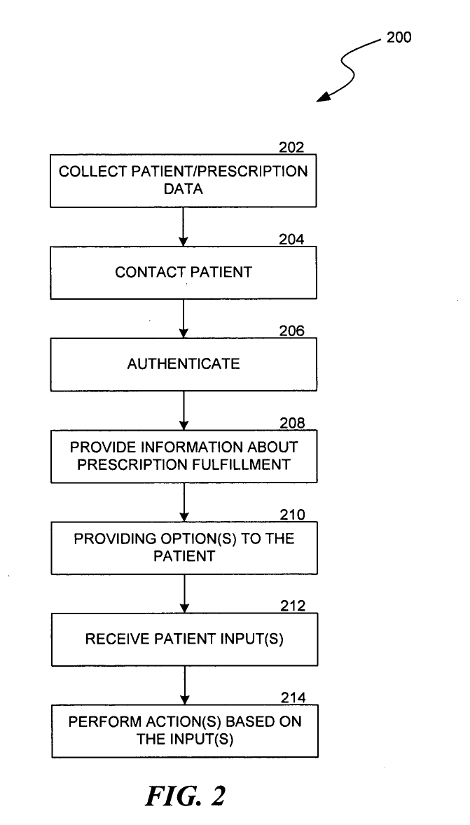

FIG. 2 is a flow diagram that illustrates a prescription management process related to prescription fulfillment 200 in accordance with various embodiments of the invention. In FIG. 2, the prescription medication fulfillment process 200 includes collecting prescription related data for one or more patients (process portion 202). For example, in certain embodiments each patient can be associated with one or more prescriptions and in the prescription related data can include the patient's identification and/or one or more drug names associated with the one or more prescriptions.

In selected embodiments, the prescription related data can be collected by a medical services facility, such as a pharmacy or doctor's office, when a patient submits a prescription for fulfillment. In certain embodiments, prescription related data collected by the medical services facility can be subsequently entered into and collected by the prescription management system. In other embodiments, prescription related information can also include other types of information and/or be obtained via other methods. For example, various types of prescription related data can include information about various medications obtained from a drug information database and/or historical patient information obtained from an insurance database.

The fulfillment process 200 can further include contacting at least one patient (process portion 204), authenticating the identity of the at least one patient (process portion 206), and providing information to the at least one patient about fulfillment of the patient's one or more prescriptions (process portion 208). For example, in certain embodiments providing information to the patient(s) can include providing information about a time to refill one or more prescriptions, a delay in filling one or more prescriptions, a recall of one or more prescriptions, a generic substitution of one or more prescriptions, a generic substitution option regarding one or more prescriptions, the status of the fulfillment of one or more prescriptions, and/or a notice regarding a last refill on one or more prescriptions. In other embodiments, providing information can include an offer to enroll patient(s) in an automatic refill program that automatically refills the patient(s) one or more prescriptions when the patient(s) is expected to run out of medication. In other embodiments, providing information can include providing notice that a filled prescription is ready for pickup, or that the filled prescription will be ready for pickup during a selected period of time. Additionally, in certain embodiments providing information can include providing a patient with the option to have a filled prescription delivered.

In other embodiments, the fulfillment process 200 can include providing the patient(s) with one or more response options regarding the fulfillment of one or more prescriptions (process portion 210) and receiving an input indicating a selection of at least one of the one or more response options (process portion 212). In still other embodiments, the fulfillment process 200 can include performing an action based on the input received (process portion 214). For example, in certain embodiments the input indicating a selection of one or more response options can be reported or sent to a pharmacy, pharmacist, pharmacy staff member, and/or other medical service provider (e.g., a doctor, doctor's office, or doctor's staff). In still other embodiments, the input indicating a selection of one or more response options can be stored for future use (e.g., to send a reminder to a pharmacist or pharmacy staff member that it is time to refill a prescription based on an automatic refill selection). In various embodiments, some, all, or none of the process portions discussed above can be performed in a computing environment.

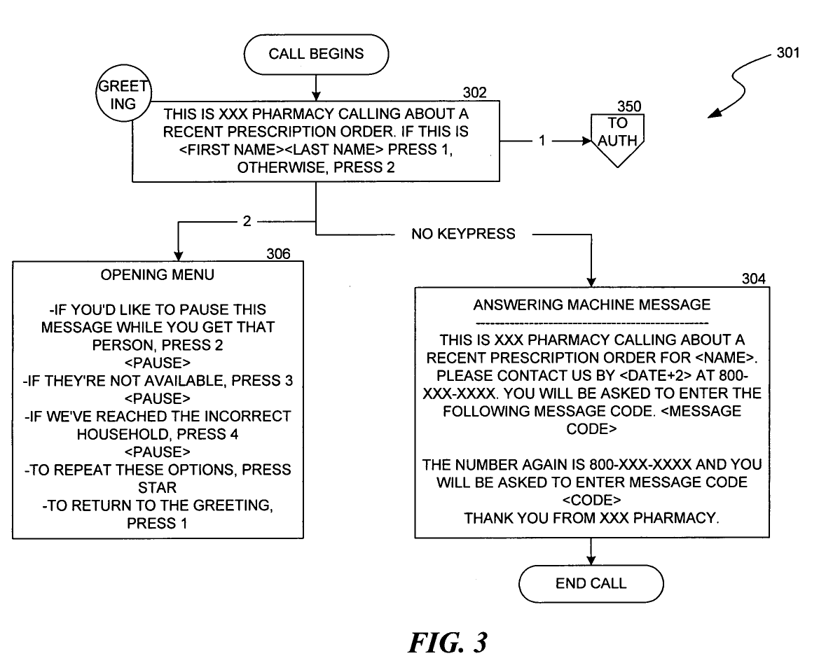

FIG. 3 is a flow diagram that illustrates a portion of a prescription management process related to prescription fulfillment in accordance with selected embodiments of the invention. In FIG. 3, prescription related data has been collected and a phone call is placed to a patient. For example, the phone call can be placed by a prescription management system that includes a computing system similar to the computing system 100 discussed above with reference to FIG. 1. When the phone call is answered, the greeting process 301 begins by playing a greeting message (process portion 302) that provides the person answering the phone with an option to transfer to an authentication process 350 via a first phone key selection or to transfer to an opening menu process 306 via a second phone key selection. If there is no key selection, a message requesting that the patient contact the pharmacy and/or a prescription management system (e.g., via phone) is played for recordation on an answering machine (process portion 304). From the opening menu process 306 the person answering the phone can select multiple response options, including returning to the greeting message.

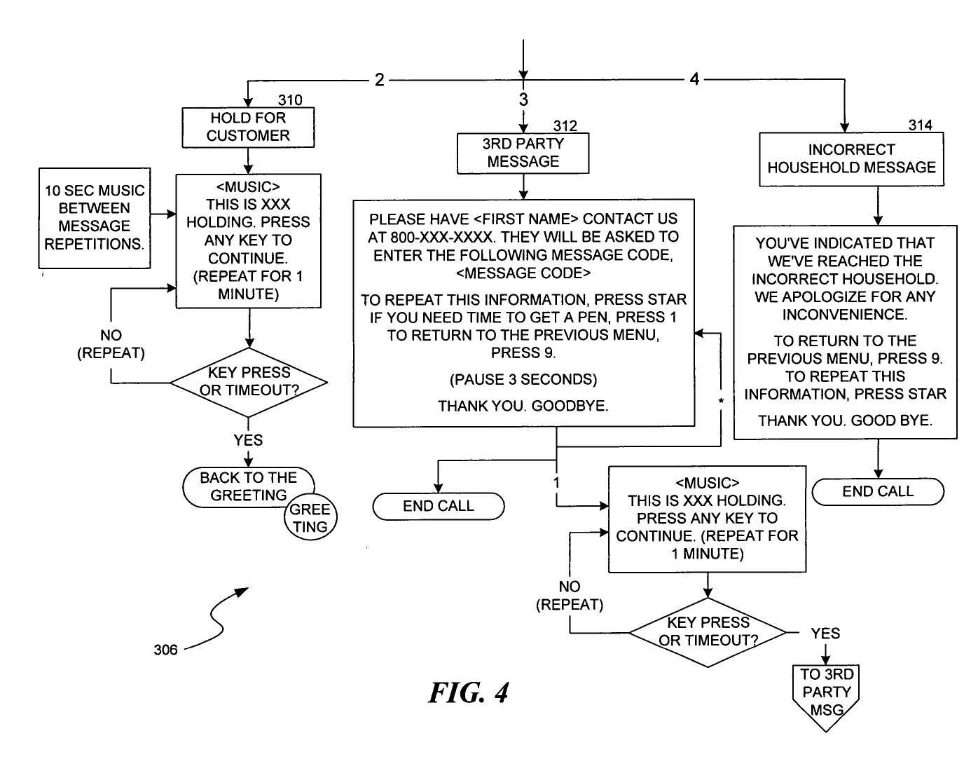

FIG. 4 is a flow diagram that illustrates additional portions of the opening menu process 306 corresponding to various phone key selections shown in FIG. 3. For example, if the person answering the phone selects “2” in the opening menu process 306, shown in FIG. 3, the hold process 310 is initiated allowing the person answering the phone to return to the greeting process after a selected period of time. If the person answering the phones selects “3” in the opening menu process 306, shown in FIG. 3, the third-party message process 312 is initiated and provides the person answering the phone with a message requesting that the person answering the phone to have the patient contact the pharmacy and/or a prescription management system. In certain embodiments, the third-party message process 312 and/or the message played for the answering machine can provide an access code or other identification for the patient to use when contacting the pharmacy and/or the prescription management system. If the person answering the phones selects “4” in the opening menu process, shown in FIG. 3, the incorrect household process 314 is initiated playing a message that apologizes for any inconvenience and also provides the person answering the phone the option to return to the previous menu.

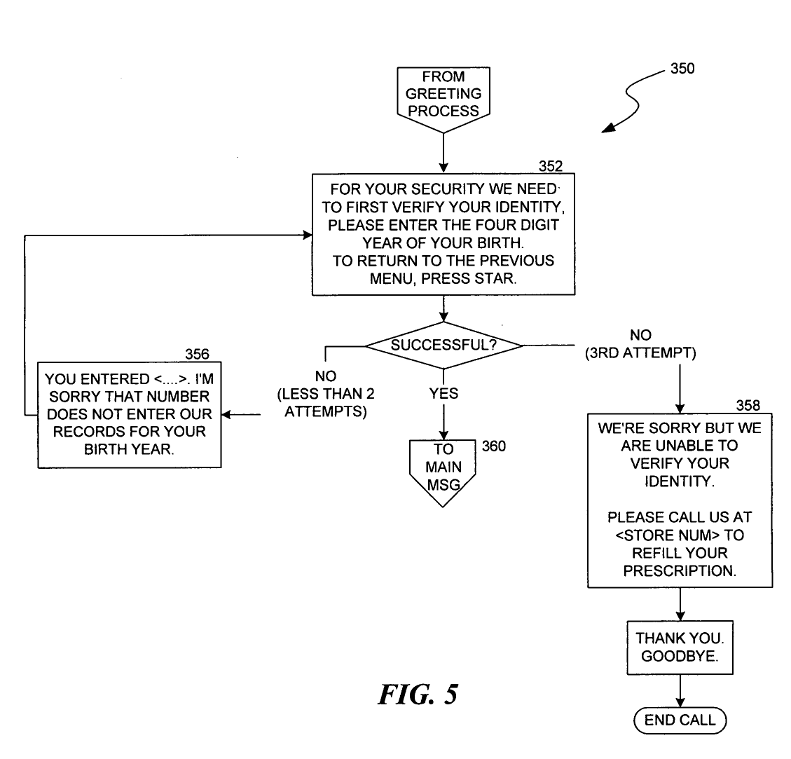

FIG. 5 is a flow diagram that illustrates the authentication process 350, discussed above with reference to FIG. 3. The authentication process 350 can be used to authenticate an identity of a patient. The authentication process can serve various functions, including providing patient privacy, providing security, and/or meeting regulatory requirements. In the illustrated embodiment, the authentication process 350 includes a request that the patient enter some form of identification (process portion 352). Various forms of identification can be used, including a birth date, a birth year, an access code, or other identifying information. Alphanumeric forms of identification can be particularly useful when inputting the identification via phone key selection. However, in other embodiments other forms of identification can be used (e.g., voice-recognition) and/or other authentication processes can be used. In still other embodiments, an authentication process is not used (e.g., when the patient is contacted via email or instant messaging).

In the illustrated embodiment, if the person answering the phone enters incorrect identification information, the person is provided with additional opportunities to enter the correct information (process portion 356). If no identification information is entered or a selected number of incorrect entries are made, the person answering the phone is provided a phone number to call for assistance (process portion 358). If the correct identification information is entered within the allowed number of attempts, the person answering the phone is transferred to the main message process 360, which can provide information and/or various options to the patient. As used herein, the term “patient” refers to a person for whom a corresponding prescription is written and/or that person's representative(s). For example, if the prescription related information includes a prescription written for a child, the patient can include the child and/or the child's parent(s) or guardian(s).

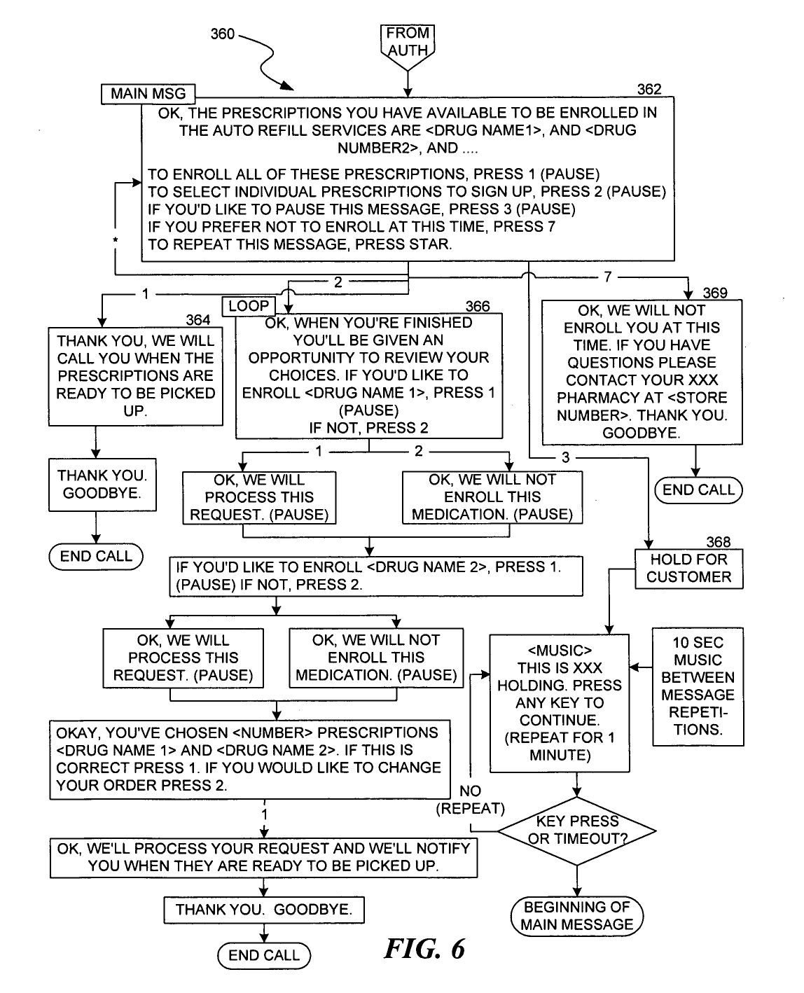

FIG. 6 is a flow diagram that illustrates an embodiment of the main message process 360 discussed above. In certain embodiments, the main message process can provide the patient with information regarding prescription fulfillment, information regarding care support information related to one or more prescriptions, and/or information regarding a request for additional information from the patient. In FIG. 6, the main message process 360 provides the patient with the opportunity to have one or more prescriptions automatically refilled at appropriate time(s) over the time period that the prescription is valid For example, the prescription related data can include one or more new prescriptions for a patient that have been received by a pharmacy and the prescription management system can contact the patient to see if the patient would like to have one or more of the new prescriptions automatically refilled when the patient would be expected to have run out of the corresponding medication (e.g., once a month or once every 90 days depending on the quantity dispensed each time the prescription is filled).

In the illustrated embodiment, the main message process 360 includes a main message 362 that informs the patient that one or more prescriptions are eligible for enrollment in an automatic refill program and provide the patient with various response options that are selectable by pressing one or more phone keys. For example, in FIG. 6 the patient can indicate that the patient wishes to have all eligible prescriptions enrolled in the automatic refill service (process portion 364) or none of the eligible prescriptions enrolled in the automatic refill service (process portion 369). The patient can also indicate that the patient wishes to have only selected eligible prescriptions enrolled in the automatic refill service (process portion 366). In the illustrated embodiment, the selection of prescriptions to be enrolled in the automatic refill program can be made via additional phone key selections in response to various options presented by the prescription management system. Additionally, in FIG. 6 the patient can pause the message (process portion 368), for example, in order to get pen, paper, and/or additional information necessary to make an automatic refill enrollment decision.

Once the patient selects one or more response options, the prescription management system can take one or more additional actions. For example, the patient's response options selections can be stored, processed, and/or reported to an entity, such as a pharmacy or a pharmacy staff member (including a pharmacist). In certain embodiments, the automatic refill enrollment of a selected prescription can be processed and the prescription management system can provide a reminder (e.g., to a pharmacy) when it is time to refill the selected prescription.

Although in FIG. 6 the main message provides a patient with the ability to enroll eligible prescription(s) in an automatic refill service, in other embodiments the main message can provide other kinds of information. For example, in selected embodiments the main message can inform the patient that the doctor has authorized a generic substitution for the prescribed medication and that the prescription will be filled with the generic drug. In other embodiments, the main message can provide the patient with a selectable response option to have a generic drug substituted, if authorized by the prescription, and/or to request that the pharmacy contact the prescribing physician to request a generic substitution.

In still other embodiments, the main message can “thank” the patient for placing a new prescription order with the pharmacy, offer the patient the option to enroll in the automatic refill service, and provide options regarding care support services and/or patient opinion information (both discussed below in further detail). For example, in certain embodiments the main message can offer the patient the option to enroll in the automatic refill service along with the option to be immediately connected with a pharmacist for a consultation regarding the prescription and/or the option to complete a survey related to the prescription. The survey can query the patient for information related to the prescription (e.g., concerns about drug side effects, concerns about prescription costs, and/or concerns about the relationship of the drug to a specific illness). Based on the patient's response to the survey, the pharmacist can call the patient to discuss specific issues. This feature can be especially useful for serving new therapy patients. Additionally, if the patient declines to have the prescription automatically refilled, the patient can be provided additional survey questions regarding the reasons for declining this option. In yet other embodiments, the prescription management system can provide still other information.

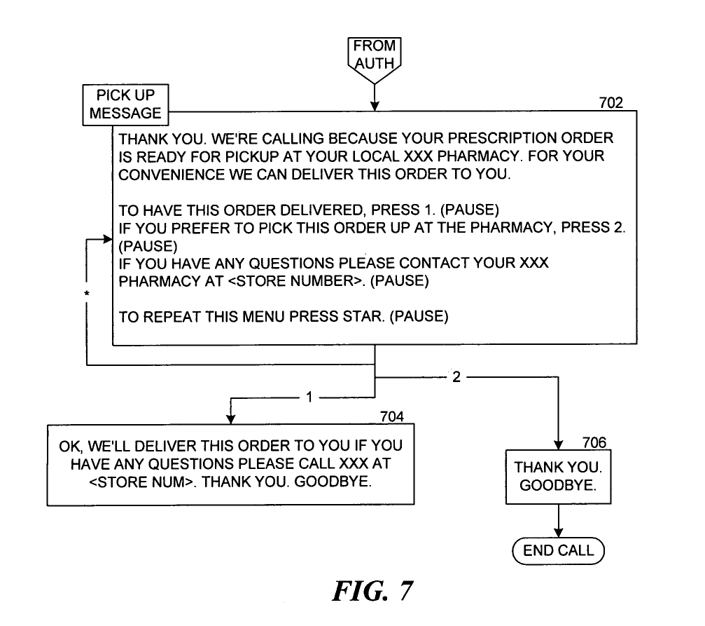

For example, FIG. 7 is a flow diagram that illustrates a portion of a prescription management process related to prescription fulfillment in accordance with still other embodiments of the invention. In FIG. 7, after a patient has been authenticated, a pickup message (e.g., similar to the main message discussed above with reference to FIG. 6) is provided to the patient (process portion 702) to inform the patient that one or more prescriptions have been filled and are ready for pickup and/or delivered. In selected embodiments, the pickup message can provide the patient with the option to indicate that the one or more filled prescriptions will be picked up at the pharmacy or to have the one or more filled prescriptions delivered. In other embodiments, the pickup message simply informs the patient that the prescription is ready for pickup. In selected embodiments, the selected response option can be reported to the corresponding pharmacy so that the pharmacy can take appropriate action.

In the illustrated embodiment, if the patient indicates that the prescription will be picked up at the pharmacy the prescription management system thanks the patient and ends the call (process portion 706). If the patient selects the delivery option, the prescription management system provides confirmation that the prescription will be delivered (process portion 704). In certain embodiments, if the patient selects the delivery option, the prescription management system can provide additional response options related to the payment method the patient expects to use when the medication is delivered.

The pickup message, shown in FIG. 7, can be used to indicate that a filled prescription is ready for pickup whether the prescription was filled automatically or not. In still other embodiments, the prescription management system can provide additional information and/or options associated with a filled prescription. For example, in selected embodiments the prescription management system can notify a patient that a filled prescription is ready for pickup and that the last refill of the prescription has been used. Additionally, in certain embodiments the prescription management system can also provide the patient with an option to have the pharmacy contact the patient's physician to have a filled prescription renewed (e.g., when the last refill has been used to fill the current prescription).

In still other embodiments, a pickup message can inform a patient of the hours during which the pharmacy open and during which a filled prescription is available for pickup. Additionally, in certain embodiments the pickup message can provide a patient with a store location and/or a store phone number. In yet other embodiments, the pickup message can inform a patient that a prescription will be available for pickup only during a selected period of time. In still other embodiments, when a filled prescription has not been picked up during a first selected period of time, the prescription management system can notify a patient that the filled prescription will only be available for pickup during a second selected period of time, and if the prescription is not picked up during the second selected period of time, the patient will have to place a new request with the pharmacy for the prescription. Although in the illustrated embodiment, the pickup message 702 is provided to the patient after patient authentication, in other embodiments the prescription management system calls the patient and provides the associated pickup information without patient authentication. In still other embodiments, the prescription management system can provide other types of information to the patient.

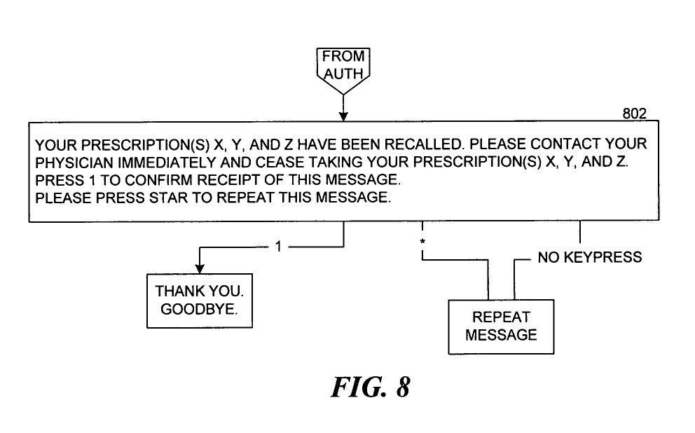

For example, FIG. 8 is a flow diagram that illustrates a portion of a prescription management process related to prescription fulfillment in accordance with yet other embodiments of the invention. In FIG. 8, after patient authentication, an informational message 802, similar to the main message discussed above with reference to FIG. 6, is provided to inform a patient that one or more prescription medications associated with the patient have been recalled. In certain embodiments, the informational message 802 can provide additional information and/or one or more options. For example, in the illustrated embodiment the informational message 802 provides a response option for use by the patient to confirm receipt of the recall notification. Additionally, in the illustrated embodiment, the informational message includes instructions to cease taking the medication and to contact the patient's physician immediately. In other embodiments, other instructions can be provided. In selected embodiments, the prescription management system can track the receipt of the recall notification and provide a summary of information to the corresponding pharmacy.

In still other embodiments, the informational message 802 to can include other types of information. For example, the informational message can include notification that it is time to fill a prescription, that fulfillment of a prescription has been delayed, a status of a prescription (e.g., information that the pharmacy has contacted the patient's physician about a generic substitution but the physician has not yet responded), a separate independent notice that there are no refills available on a selected prescription (e.g., because the authorized number of refills has been reached and/or a selected time period has expired), and/or information regarding the potential substitution of various generic drugs.

As discussed above, the prescription management system can include a computing system and some, all, or none of the prescription management process can be performed in a computing environment. Additionally, although embodiments of the prescription management method discussed above with reference to FIGS. 3-8 have been described using a phone key selection interface to receive patient selections, those skilled in the art will understand that in other embodiments other methods of interfacing with the patient can be used. For example, in other embodiments the prescription management system can use voice recognition, email, fax, websites, and/or instant messaging to interface with patients.

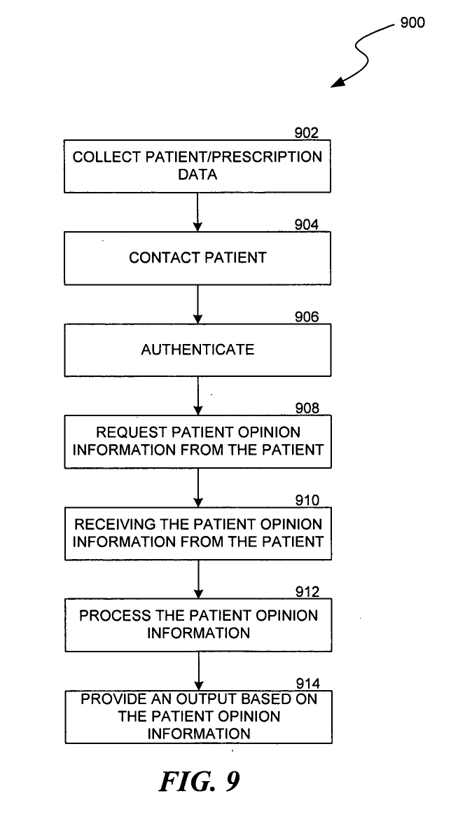

FIG. 9 is a flow diagram that illustrates a prescription management process related to collecting patient opinion information from a patient associated with a medication 900 in accordance embodiments of the invention. The process in FIG. 9 can include collecting prescription related data for each one of multiple patients (process portion 902) and contacting at least one of the patients (process portion 904). Each patient can be associated with one or more prescriptions and prescription related data can include at least one of the patient's identification and one or more drug names associated with the one or more prescriptions. The process 900 can further include authenticating an identity of at least one patient (process portion 906). In certain embodiments, the process of collecting prescription related data, the process of contacting at least one patient, and the process of the authenticating an identity of the at least one patient can be similar to the corresponding processes discussed above with reference to FIGS. 3-8.

The process 900 can further include providing information that requests patient opinion information or data associated with a prescription medication from the at least one patient (process portion 908). For example, in certain embodiments requesting patient opinion information can include a request for feedback about a pharmacy, a service provided by a pharmacy, a medical service related to one or more prescriptions, and/or a patient's decision to have a prescription filled or not filled. In various embodiments, the request for patient opinion information can include one or more response options presented to the patient.

The process 900 can also include receiving the patient opinion information from the at least one of the patient (process portion 910). For example, receiving the patient opinion information can include receiving an input indicating a selection of one or more response options presented to the patient. The process 900 can further include processing the patient opinion information (process portion 912). For example, the patient opinion information received from one or more patients can be summarized and/or analyzed to provide a more meaningful format or output. In one embodiment, the reasons that patients do not have prescriptions refilled at a selected pharmacy can be tabulated to show the various reasons and the number of patients corresponding to each reason. In still other embodiments, the process 900 can include providing an output based on the patient opinion information (process portion 914). For example, the various patient inputs and/or the processed data based on patient input can be provided to a corresponding pharmacy that has filled a related prescription.

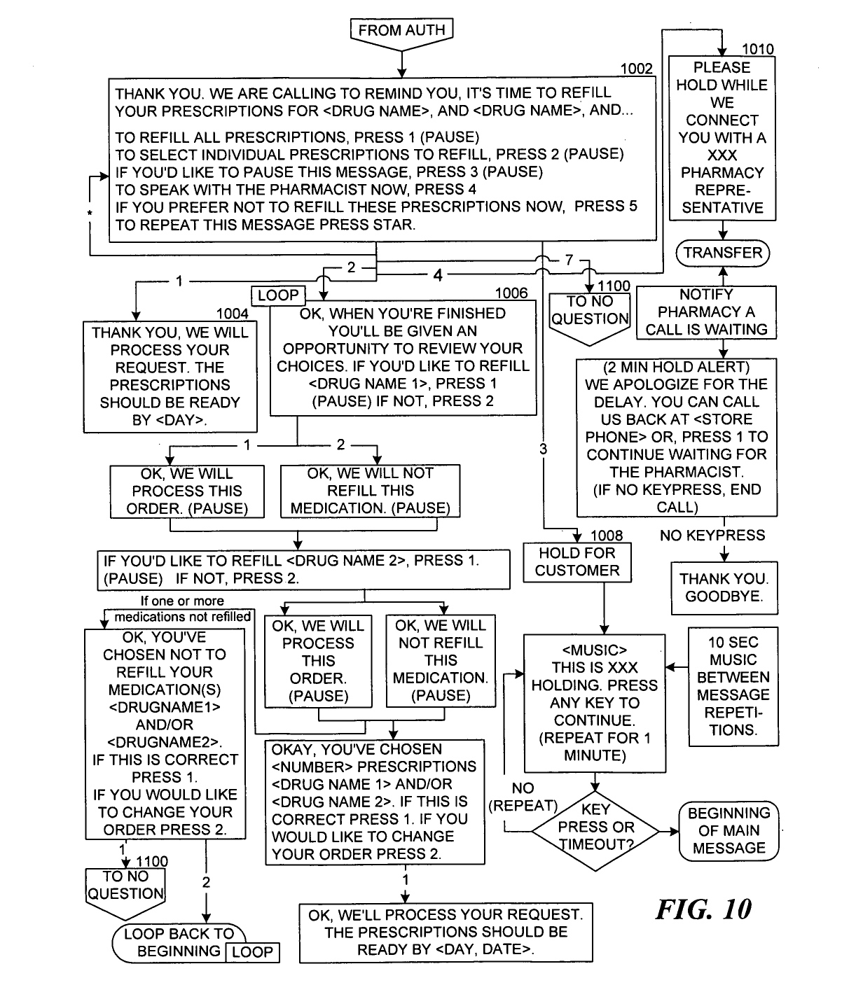

For the purpose of illustration, selected embodiments of a process for collecting patient opinion information will be discussed in combination with a process for reminding a patient to refill a prescription. It will, however, be understood by those skilled in the art that these two processes can be practiced individually and/or combinations with other processes is discussed herein. FIG. 10 is a flow diagram that illustrates a portion of a prescription management process that is related to providing information that reminds a patient to refill a prescription and requests patient opinion information associated with a medication in accordance with various embodiments of the invention. In FIG. 10, after a patient has been contacted and authenticated (e.g., similar to the process is discussed above with reference to FIGS. 3-8), the prescription management system can provide a main message reminding a patient that it is time to refill a prescription 1002. In certain embodiments, the reminder message 1002 can include one or more response options allowing the patient to have one or more prescriptions filled. For Example, in FIG. 10 the patient can select various response options via a key press on a phone. The patient can choose to have all prescriptions refilled (process portion 1004), selected prescriptions refilled (process portion 1006), or no prescriptions refilled. Additionally, in the illustrated embodiment the patient can pause the message (process portion 1008) and/or request to be connected to a pharmacy staff member (process portion 1010). In FIG. 10, if the patient chooses to not refill one or more prescriptions, a patient enters the no question process 1100.

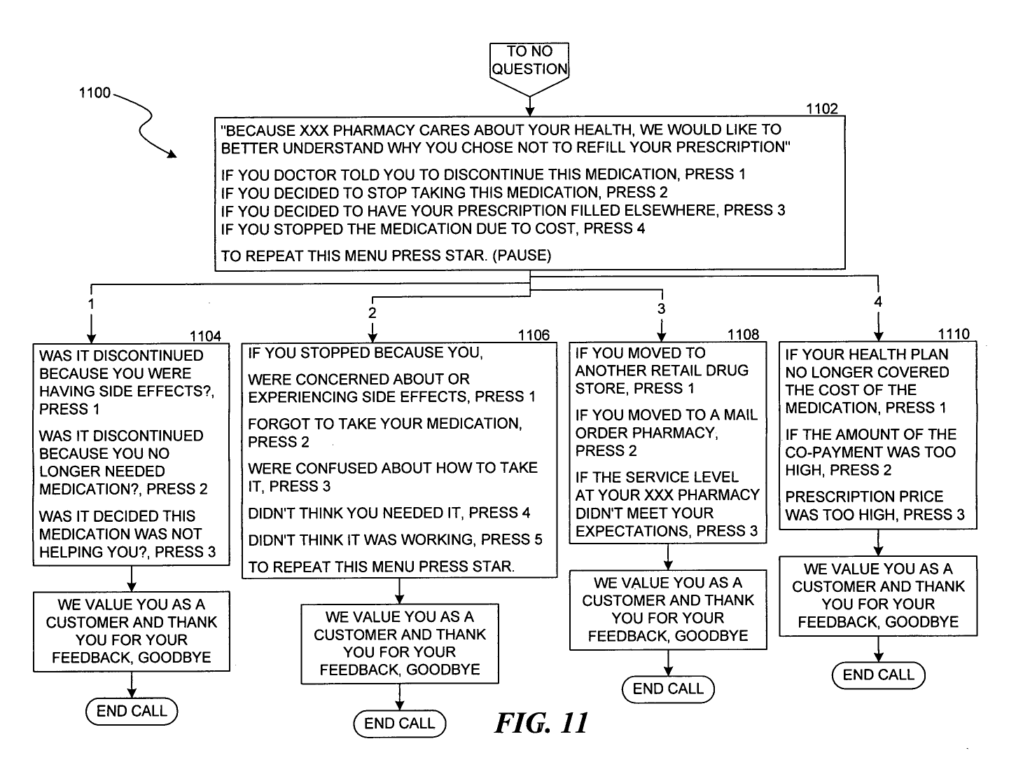

FIG. 11 is a flow diagram that illustrates the no question process 1100 in accordance various embodiments of the invention. In FIG. 11, the no question process 1100 includes a request for patient opinion information (process portion 1102) and provides a patient with one or more response options. If the patient selects the response option indicating that the doctor instructed the patient to discontinue the medication, the patient is presented with the doctor response process 1104. The doctor response process 1104 requests additional information from the patient and includes one or more response options to facilitate patient opinion information collection. If the patient indicates that it was the patient's decision to stop taking the medication, the patient is present with the patient response process 1106, which requests additional data. If the patient indicates that the patient has decided to have the prescription filled at a different pharmacy, the patient is presented with the pharmacy information process 1108, which requests opinion data regarding the original pharmacy. If the patient indicates that the patient has stopped taking the medication due to cost, the patient is presented with the cost information process 1110, which requests information regarding the cost of the medication. In other embodiments, the prescription management system can collect other types of information regarding patient opinion.

As discussed above, the prescription management system can include a computing system and some, all, or none of the information collection process discussed above with reference to FIGS. 9-11 can be performed in a computing environment. Additionally, although embodiments of the information collection process discussed above with reference to FIGS. 10-11 have been described using a phone key selection interface to receive patient input, those skilled in the art will understand that in other embodiments other methods of interfacing with the patient can be used. For example, in other embodiments the prescription management system can use voice recognition, email, fax, websites, and/or instant messaging to interface with patients.

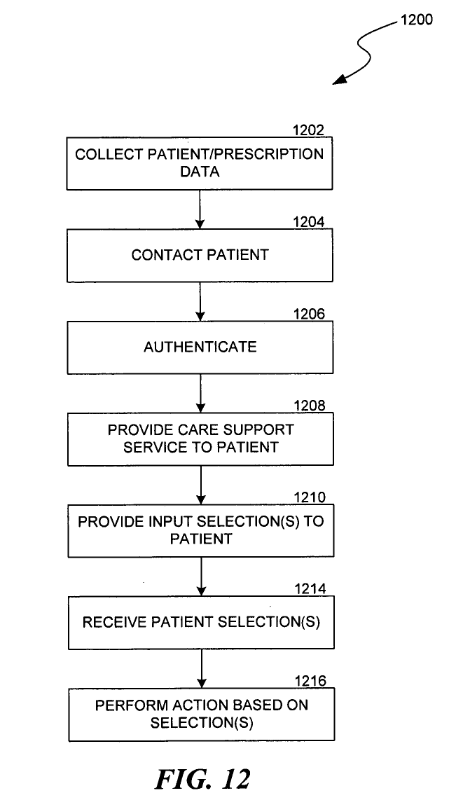

FIG. 12 is a flow diagram that illustrates a prescription management process for providing care support services related to prescription medication 1200 in accordance with embodiments of the invention. The process 1200 in FIG. 12 can include collecting prescription related data for each one of multiple patients (process portion 1202) and contacting at least one of the patients (process portion 1204). Each patient can be associated with one or more prescriptions and prescription related data can include at least one of the patient's identification and one or more drug names associated with the one or more prescriptions. The process 1200 can further include authenticating an identity of at least one patient (process portion 1206). In certain embodiments, the process of collecting prescription related data, the process of contacting at least one patient, and the process of the authenticating an identity of the at least one patient can be similar to the corresponding processes discussed above with reference to FIGS. 3-8.

In the illustrated embodiment, the process 1200 further includes providing care support information to the at least one patient. In certain embodiments, the care support information can be related to the patient's one or more associated prescriptions. For example, the care support service information provided to the at least one patient can include, among other things, providing new medication counseling, a reminder to make a follow-up medical appointment related to a medication, a reminder to take a medication, a tip on how to remember taking a medication, health education related to a prescribed medication, testimonial information about a personal experience related to a medication or related medical condition, information related to drug dosage adherence monitoring, patient monitoring related to a medication, automatic consultation scheduling (e.g., scheduling an appointments with a medical service provider), phone reminders regarding scheduled consultation appointments, and/or information related to drug side effects. In certain embodiments, the care support information can include various response options that allow patient interaction so that the care support service information can be better tailored to the patient's needs.

In selected embodiments, the process 1200 can further include a request for input and/or one or more response options (process portion 1210). In other embodiments, the process 1200 can still further include receiving one or more inputs from the patient indicating a selection of one or more of the response options (process portion 214). It still other embodiments, the process 1200 can further include performing an action based on one or more inputs received from the patient (process portion 216). For example, in certain embodiments one or more of the inputs received from the at least one patient can be provided to a medical staff member (e.g., a pharmacy staff member and/or staff member had another type of medical facility).

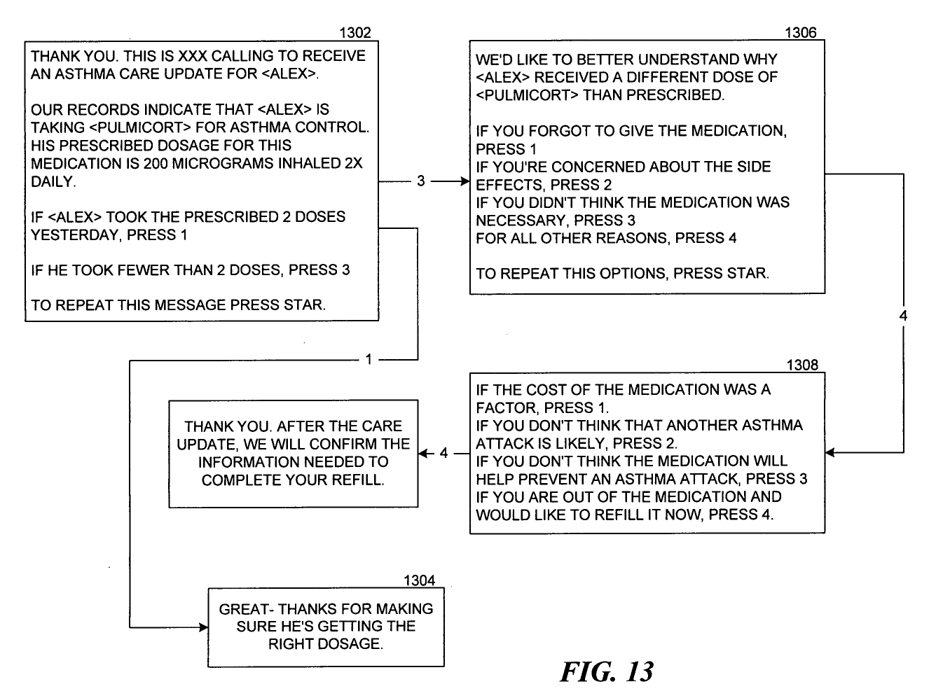

FIGS. 13-15 illustrate various embodiments of the Process 1200, shown in FIG. 12. Although in the illustrated embodiment these processes are shown linked together, in other embodiments the processes discussed in FIGS. 13-15 can stand alone or be combined with other processes described herein. FIG. 13 is a flow diagram that illustrates a portion of a prescription management process for providing care support services related to prescription medication in accordance with various embodiments of the invention. In FIG. 13, the patient (e.g., the mother of the person for whom the prescription is written) is provided information regarding the prescription and requested to respond to multiple response options (process portion 1302). If the patient indicates (e.g., via telephone key presses) that the prescribed dosage has been taken, the prescription management system provides acknowledgment (process portion 1304). If the patient indicates that less than the prescribed dosage was taken, the process management system queries the patient as to why a lesser dosage was taken in process portions 1306 and 1308. In certain embodiments, the patient input received can be provided to a pharmacy or other medical service provider. Accordingly, medication dosage adherence can be monitored and assistance can be provided where necessary. Additionally, this process can serve as a reminder to take the medication as prescribed.

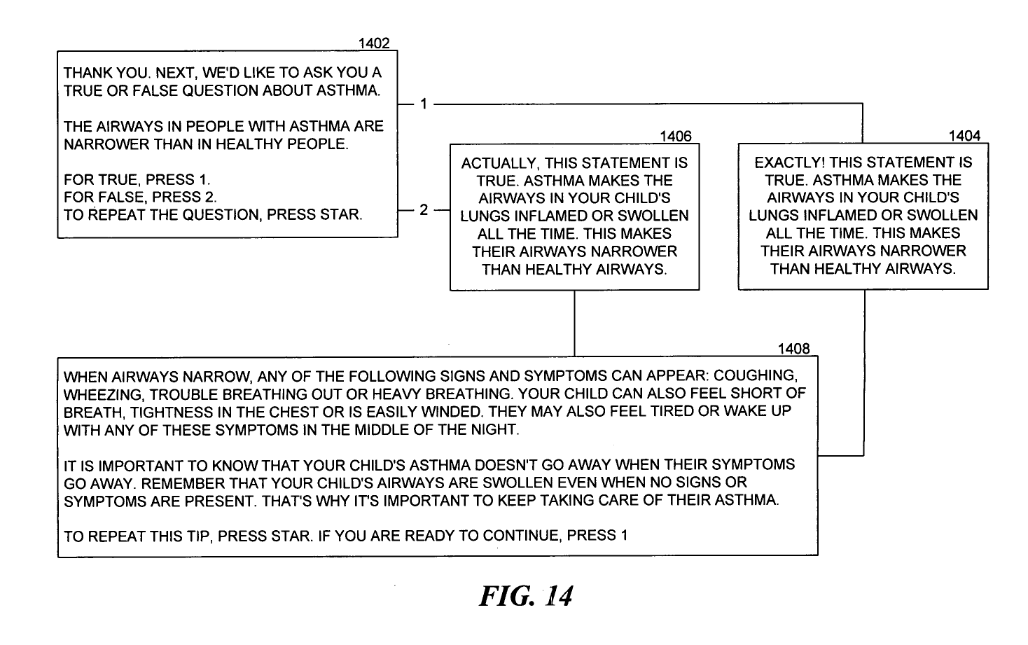

In FIG. 14 the prescription management system queries a patient about general medical information related to the prescription that has been prescribed (process 1402). The patient is then provided feedback depending on the response option the patient selects (process portions 1404 and 1406). In either case, the patient is provided with additional information regarding additional general medical information related to the prescription (process portion 1408). Accordingly, the patient receives health education about a general medical condition related to a prescription.

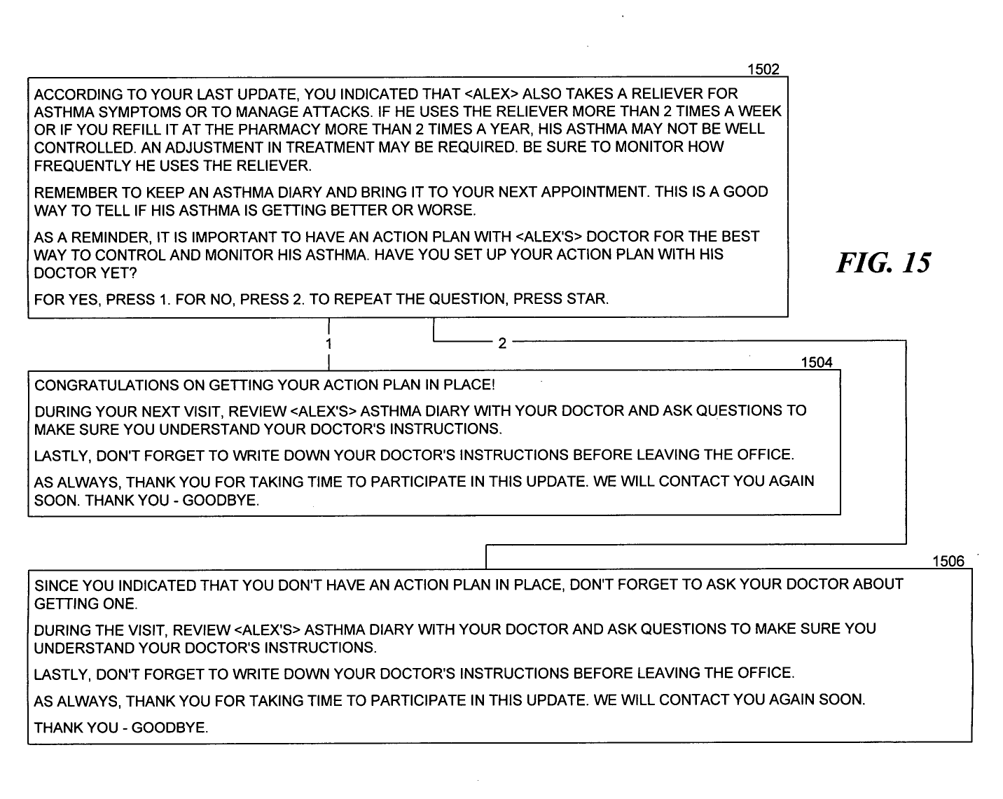

In FIG. 15 the prescription management systems queries the patient about additional information related to the prescription and recommends follow-up medical services (process portion 1502). Depending on the response option selected by the patient, the prescription management system provides tailored information regarding further instructions (process portions 1504 and 1506). Accordingly, the patient receives a reminder regarding follow-up medical care related to the prescription. In other embodiments, the prescription management system can provide information related to newly prescribed medications (e.g., prescription counseling) and/or tips for remembering to take medication (e.g., a suggestion to keep the medication by the patient's toothbrush to aid the patient in remembering to take the medication). In still other embodiments, the prescription management system can provide information and/or elicit responses to aid in patient monitoring. For example, in certain embodiments the prescription management system can periodically contact a patient and provide a series of phone key selectable response options to determine the patient's mood (e.g., when the patient is taking a medication related to depression). In other embodiments, the prescription management system can provide response options where the patient input will provide an indication of the presence or absence of drug related side effects.

As discussed above, the prescription management system can include a computing system and some, all, or none of the care support service processes discussed above with reference to FIGS. 12-15 can be performed in a computing environment. Additionally, although embodiments of the care support service process discussed above with reference to FIGS. 13-15 have been described using a phone key selection interface to receive input from the patient, those skilled in the art will understand that in other embodiments other methods of interfacing with the client can be used. For example, in other embodiments the prescription management system can use voice recognition, email, fax, websites, and/or instant messaging to interface with patients.

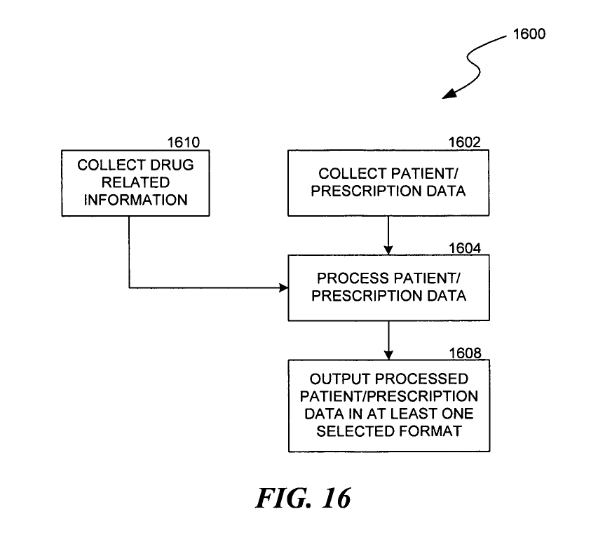

FIG. 16 is a flow diagram that illustrates a process for providing formatted prescription related data for use in a prescription management element 1600 in accordance with still other embodiments of the invention. For example, in certain embodiments a prescription management system can include a prescription management element and an interface element. The interface element can collect raw or unformatted prescription related information (e.g., from an existing pharmacy database), process the information, and provide the formatted prescription related information to the prescription management element. The prescription management element can then contact one or more patients and can provide prescription related information to the patients (e.g., as discussed above with reference to FIGS. 2-15). For example, the prescription management element can provide prescription fulfillment information, provides care support service information, and/or request patient opinion information.

In the illustrated embodiment, the process 1600 includes collecting raw or unformatted prescription related data for each one of multiple patients (process portion 1602). Each patient can be associated with one or more prescriptions and prescription related data can include at least one of the patient's identification and one or more drug names associated with the one or more prescriptions. Portions of the raw prescription related data can come from one or more sources and can have various formats. For example, the raw prescription related data can be collected from multiple pharmacies, multiple medical service providers/facilities, multiple databases, and/or multiple prescription related systems. In selected embodiments, at least a portion of the raw prescription related data can come from various existing (e.g., legacy) pharmacy systems or databases, such as TechRX™, available from the NDCHealth®) Corporation of Atlanta, Ga.; PDX, available from PDX, Inc. of Fort Worth, Tex.; Synercom™, available from HCC of Fort Worth, Tex.; and ScriptPro®, available from ScriptPro LLC of Mission Kans.

Because the raw prescription related data may not be in a format that is usable by a prescription management element, it can be necessary to process the raw prescription related data in order to place the data in a format that the prescription management element can use. Accordingly, the process 1600 can include processing the raw prescription related data (process portion 1604) to provide formatted prescription related data that is usable by a prescription management element. In certain embodiments, processing the prescription related data can include combining various portions of prescription related data, aggregating a portion of prescription related data, parsing a portion of the prescription related data, summarizing a portion of prescription related data, coding the prescription related data to be readable/usable by a prescription management element, and/or filtering a portion of prescription related data. Filtering prescription related data can include various functions, such as removing duplicate data entries and/or scrubbing the data to remove extraneous information.

In other embodiments, the process 1600 can include collecting drug-related information (process portion 1610) and the drug-related information can be used to supplement and/or filter the raw prescription related data. For example, in certain embodiments, during prescription related data processing the prescription related data can be filtered against the drug related information. For instance, the prescription related data can be filtered against the drug-related information to determine an availability of a drug associated with one or more of the prescriptions, a cost of the drug associated with one or more the prescriptions, an availability of a generic drug related to a medication associated with one or more of the prescriptions, a cost of a generic drug related to a medication associated with one or more of the prescriptions, whether a drug associated with one or more of the prescriptions is being recalled, a side effect associated with the drug associated with one or more the prescriptions, and/or a potential for interaction between a first prescription associated with a selected patient in a second prescription associated with the selected patient.

The formatted prescription related data can then be provided for use by a prescription management element (process portion 1608). For instance, the process 1600 can be used to provide formatted prescription related data to a prescription management element that provides prescription fulfillment information, provides care support service information, and/or collects patient opinion information. Additionally, as discussed above, the prescription management system can include a computing system and some, all, or none of the processes for providing formatted prescription related data for use in a prescription management element (discussed above with reference to FIG. 16) can be performed in a computing environment.

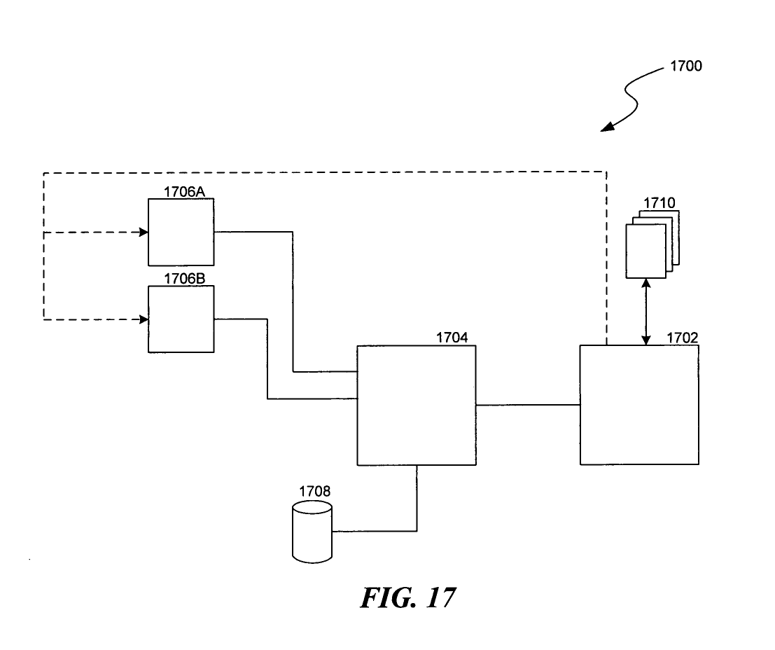

FIG. 17 is a partially schematic illustration of a prescription management system 1700 suitable for implementing various embodiments of the invention. In FIG. 17, the prescription management system 1700 includes a prescription management element 1702 and an interface element 1704. The prescription management element 1702 can be configured to communicate with one or more patients 1710 (e.g., via a patient's phone system and/or computing system). Each patient can be associated with one or more prescriptions, and the prescription management element 1702 can provide information to, and/or collect information from, the patient's 1710. For example, in selected embodiments the prescription management element can be configured to provide prescription fulfillment information, provide care support service information, and/or provide information requesting patient opinion information. As discussed above, in various embodiments the raw prescription related data and/or the formatted prescription related data can include at least one of the patient's identification associated with one or more prescriptions and one or more drug names associated with the one or more prescriptions.

The interface element 1704 can be operatively coupled to the prescription management element 1702 to provide formatted prescription related data to the prescription management element 1702. The interface element 1704 can include a computing element or system that is configured to collect raw or unformatted prescription related data that would be unusable by the prescription management element, process the raw prescription related data, and provide formatted prescription related data to the prescription management element 1702 (e.g., formatted prescription related data that is formatted to be usable by the prescription management element 1702). For example, in various embodiments the interface elements 1704 can process the raw prescription related data as discussed above with reference to FIG. 16.

As discussed above, the raw prescription related data can be gathered from various and/or multiple sources (e.g., from existing or legacy databases of various medical service providers). For Example, in FIG. 17 two pharmacies (shown as a first pharmacy 1706a and a second pharmacy 1706b) are shown coupled to the interface element 1704 to provide raw prescription related data to the prescription management system 1700. Additionally, in the illustrated embodiment the prescription management element 1702 of the prescription management system 1700 is configured to provide output (e.g., information or data) to the pharmacies 1706a and 1706b (e.g., based on patient selections).

For example, as discussed above, in certain embodiments the prescription management system 1700 can provide raw data collected by the system and/or analytics of the collected data to a medical service provider. This information or data can be used for various purposes including providing information that will allow the medical service provider to better serve patient needs and/or identify areas where the medical service provider needs to improve performance. Additionally, providing data analytics can include providing formatted or summarized information. For example, in certain embodiments the data analytics can provide information to a medical service provider that is summarized according to various demographics (e.g., age, race, sex, drug, insurance coverage, and/or medical condition) and/or other common characteristics.

In certain embodiments, the interface element 1704 can also collect drug-related information from various databases. For example, in certain embodiments the interface element 1704 can collect data from a drug information service such as Medi-Span, available form Wolters Kluwer Health, Inc. of Indianapolis, Ind. As discussed above with reference to FIG. 16, the drug-related information can be used to supplement and/or filter the raw prescription related data.

A feature of embodiments discussed above is that a prescription management system can provide an effective and efficient way to accomplish many of the day-to-day pharmacy tasks that are time-consuming and expensive. Accordingly, selected embodiments of a prescription management system can reduce pharmacy operating costs, allow a pharmacy to provide a higher quality of service to its patients, and/or allow a pharmacy to service a larger number of patients. This feature not only can result in a cost savings and/or better health care services, but can provide pharmacy staff members with higher levels of job satisfaction because they can more effectively focus on patient needs.

From the foregoing, it will be appreciated that specific embodiments of the invention have been described herein for purposes of illustration, but that various modifications may be made without deviating from the invention. Additionally, aspects of the invention described in the context of particular embodiments may be combined or eliminated in other embodiments. For example, although advantages associated with certain embodiments of the invention have been described in the context of those embodiments, other embodiments may also exhibit such advantages. Additionally, not all embodiments need necessarily exhibit such advantages to fall within the scope of the invention. Accordingly, the invention is not limited except as by the appended claims.

I/We claim:

1. A method for providing care support services related to prescription medication, comprising:
collecting prescription related data for each one of multiple patients, each patient being associated with one or more prescriptions, the prescription related data including at least one of a patient identification and one or more drug names associated with the one or more prescriptions;
contacting at least one of the patients; and
providing one or more response options to the at least one of the patient regarding an actual dosing of a medication corresponding one or more of the at least one of the patients' associated prescriptions;
receiving an input indicating a selection of at least one of the one or more response options; and
providing information regarding the selection of at least one of the one or more response options to a medical staff member.

2. The method of claim 1 wherein a method for providing care support services related to prescription medication includes a method in a computing environment.

3. The method of claim 1 wherein a method for providing care support services related to prescription medication includes a method in a computing environment, and wherein:
contacting at least one of the patients includes contacting at least one of the patients via phone;
providing one or more response options includes providing one or more response options via phone; and
receiving an input includes receiving an input via phone.

4. The method of claim 1 wherein a method for providing care support services related to prescription medication includes a method in a computing environment, and wherein:
contacting at least one of the patients includes contacting at least one of the patients via phone;
providing one or more response options includes providing one or more response options via phone, the one or more response options being selectable via a phone key selection; and
receiving an input includes receiving an input via phone.

5. The method of claim 1, further comprising authenticating an identity of the at least one of the patients.

6. A method for providing care support services related to prescription medication, comprising:
collecting prescription related data for each one of multiple patients, each patient being associated with one or more prescriptions, the prescription related data including at least one of a patient identification and one or more drug names associated with the one or more prescriptions;
contacting at least one of the patients;
providing one or more response options regarding the at least one of the patient's condition, the at least one of the patient's condition being related to the one or more prescriptions associated with the at least one of the patients;
receiving an input indicating a selection of at least one of the one or more response options; and
providing information regarding the selection of at least one of the one or more response options to a medical staff member.

7. The method of claim 6 wherein a method for providing care support services related to prescription medication includes a method in a computing environment.

8. The method of claim 6 wherein a method for providing care support services related to prescription medication includes a method in a computing environment, and wherein:
contacting at least one of the patients includes contacting at least one of the patients via phone; and
providing one or more response options includes providing one or more response options via phone; and
receiving an input includes receiving an input via phone.

9. The method of claim 6 wherein a method for providing care support services related to prescription medication includes a method in a computing environment, and wherein:
contacting at least one of the patients includes contacting at least one of the patients via phone;
providing one or more response options includes providing one or more response options via phone, the one or more response options being selectable via a phone key selection; and
receiving an input includes receiving an input via phone.

10. The method of claim 6, further comprising authenticating an identity of the at least one of the patients.

11. The method of claim 6 wherein the patient's condition includes at least one of a patient's mood, a presence or non-presence of one or more selected side effects, an indication of the effectiveness or non-effectiveness of the medication, and a level of control of a chronic condition.

12. A method for providing care support services related to prescription medication, comprising:
collecting prescription related data for each one of multiple patients, each patient being associated with one or more prescriptions, the prescription related data including at least one of a patient identification and one or more drug names associated with the one or more prescriptions;
contacting at least one of the patients; and
providing a care support service to the at least one of the patients related to the at least one of the patient's one or more associated prescriptions.

13. The method of claim 12 wherein a method for providing care support services related to prescription medication includes a method in a computing environment.

14. The method of claim 12 wherein a method for providing care support services related to prescription medication includes a method in a computing environment, and wherein:
contacting at least one of the patients contacting at least one of the patient via phone; and
providing care support service to the at least one of the patients includes providing care support service to the at least one of the patients via phone.

15. The method of claim 12, further comprising authenticating an identity of at least one patient.

16. The method of claim 12 wherein providing care support service includes providing at least one of new medication counseling, a medical follow up reminder, a reminder to take a prescription, a tip for remembering to take a prescription, health education, testimonial information about a personal experience, information related to dosage adherence monitoring, information related to drug side effects, and information related to patient monitoring.

17. The method of claim 12, further comprising:
providing one or more response options related to the care support service; and
receiving an input indicating a selection of at least one of the one or more response options;

18. The method of claim 12, further comprising:
providing one or more response options related to the care support service;
receiving an input indicating a selection of at least one of the one or more response options; and
providing information regarding the selection of at least one of the one or more response options to a medical staff member.
